(19) United States
(12) Patent Application Publication
(10) Pub. No.: US 2024/0316541 A1
(43) Pub. Date: Sep. 26, 2024

(54) METAL MODIFIED ZSM-5 MOLECULAR SIEVE CATALYST, PREPARATION METHOD AND APPLICATION THEREOF

(71) Applicant: PetroChina Company Limited, Beijing (CN)

(72) Inventors: Penggang LV, Beijing (CN); Tao LIU, Beijing (CN); Zhishuang PAN, Beijing (CN); Xiaoliang HUANG, Beijing (CN); Hongchang DUAN, Beijing (CN); Yunfeng ZHENG, Beijing (CN); Jinjun CAI, Beijing (CN); Manyun WANG, Beijing (CN); Zhengguo TAN, Beijing (CN)

(21) Appl. No.: 18/674,013

(22) Filed: May 24, 2024

Related U.S. Application Data
(63) Continuation of application No. PCT/CN2022/138561, filed on Dec. 13, 2022.

(30) Foreign Application Priority Data
Sep. 30, 2022 (CN) 202211206094.9

(51) Int. Cl. B01J 29/40; B01J 37/04; B01J 37/08

(52) U.S. Cl. CPC B01J 29/40; B01J 37/04; B01J 37/08

(57) ABSTRACT

The present application provides a metal modified ZSM-5 molecular sieve catalyst and a preparation method and an application thereof, and the preparation method includes the following steps: mixing a ZSM-5 molecular sieve, tetraethyl orthosilicate, trichloroacetic acid, and water for reaction, and filtering to obtain an intermediate; mixing the intermediate, a melamine solution and a metal salt solution, drying and roasting, and obtaining the metal modified ZSM-5 molecular sieve catalyst. The prepared catalyst has the advantages of good catalytic activity, selectivity and stability, simple preparation process and easiness for operation.

### CROSS-REFERENCE TO RELATED APPLICATIONS

This application is a continuation of International Application No. PCT/CN2022/138561, filed on Dec. 13, 2022, which claims priority to Chinese Patent Application No. 202211206094.9, filed on Sep. 30, 2022. The disclosures of the aforementioned application are hereby incorporated by reference in their entireties.

### TECHNICAL FIELD

The present application relates to the technology field of catalysts and, in particular, a metal modified ZSM-5 molecular sieve catalyst, a preparation method and an application thereof.

### BACKGROUND

The ZSM-5 molecular sieves have a special pore canal structure and a higher specific surface area, and have been widely used in catalytic fields, such as a naphtha steam cracking (SC) and a refinery fluidized catalytic cracking (FCC) to produce the light olefins (C2-C4 olefins). The catalytic activity and the light olefins selectivity of ZSM-5 molecular sieves can be improved by metal modification, thereby enhancing the yield of light olefins.

The catalytic activity and the selectivity of metal modified ZSM-5 molecular sieves are related to a metal loading rate. The preparation process of the metal modified ZSM-5 molecular sieves will directly affect the metal loading rate. For example, the patent document CN109663613A discloses a metal modified ZSM-5 molecular sieve catalyst, and preparation and application thereof. The metal modification of ZSM-5 molecular sieve is carried out by hydrothermal method to realize the modification of ZSM-5 molecular sieve catalyst and enhance the utilization rate of modified metal. The patent document CN106140266A discloses a metal modified ZSM-5 molecular sieve catalyst and preparation method and application thereof, where the catalyst is prepared by modification of ZSM-5 molecular sieve by the hydrothermal method. The patent document CN104324746A discloses a metal modified ZSM-5 molecular sieve catalyst and application thereof, where the metal modified ZSM-5 molecular sieve catalyst is obtained by pretreating ZSM-5 raw powder at high temperature, roasting, performing ion exchange and roasting. However, the above methods have limited improvement on the structural stability of ZSM-5 molecular sieve. During the preparation process of the catalyst, the loaded metal is easily lost, thereby affecting the catalytic activity and the selectivity of the catalyst. In addition, with the increase of silicon-aluminum ratio of ZSM-5 molecular sieve, its hydrothermal stability is also gradually improved. However, it is difficult to synthesize the ZSM-5 molecular sieve with high silicon-aluminum ratio, and the skeleton structure of the molecular sieve is easily destroyed during the synthesis process, which affects the structural stability of ZSM-5 molecular sieves. Therefore, how to further improve the catalytic activity, the selectivity and the stability of catalyst is a technical problem which is urgent to be solved in the art.

### SUMMARY

The present application provides a preparation method of a metal modified ZSM-5 molecular sieve catalyst. The prepared catalyst has the advantages of good catalytic activity, selectivity, stability, etc., and its preparation process is simple and easy to operate.

A first aspect of the present application provides a preparation method of a metal modified ZSM-5 molecular sieve catalyst, including the following steps: mixing a ZSM-5 molecular sieve, tetraethyl orthosilicate, trichloroacetic acid, and water for reaction, and filtering to obtain an intermediate; mixing the intermediate, melamine solution and metal salt solution; and drying, roasting, and obtaining the metal modified ZSM-5 molecular sieve catalyst.

According to an embodiment of the present application, the process of mixing the intermediate, melamine solution and metal salt solution includes: first mixing the intermediate and the melamine solution and stirring for 10 min-30 min to obtain a mixed solution, and then mixing the mixed solution and the metal salt solution.

According to an embodiment of the present application, the melamine solution includes melamine and an organic solvent, where a mass ratio of the organic solvent to the melamine is (0.4-1):1.

According to an embodiment of the present application, a mass ratio of the melamine to the ZSM-5 molecular sieve is (0.03-0.3):1.

According to an embodiment of the present application, the organic solvent is selected from at least one of acetic acid and ethylene glycol.

According to an embodiment of the present application, a mass ratio of the trichloroacetic acid to the ZSM-5 molecular sieve is (0.8-3.5):1; and/or, a mass ratio of the tetraethyl orthosilicate to the ZSM-5 molecular sieve is (0.2-0.6):1.

According to an embodiment of the present application, the reaction is performed at a temperature of 80° C. for 10 min-60 min.

According to an embodiment of the present application, the drying is performed at a temperature of 100° C.-110° C. for 8h-14h; and/or, the roasting is performed at a temperature of 480° C.-580° C. for 1h-4h.

A second aspect of the present application provides a metal modified ZSM-5 molecular sieve catalyst, which is prepared by the above-mentioned preparation method.

A third aspect of the present application provides a preparation method of light olefins, including: allowing alkane material to react under a catalytic action of the above-mentioned metal modified ZSM-5 molecular sieve catalyst to produce the light olefins.

The implementation of the present application has at least the following beneficial effects.

For the preparation method of the metal modified ZSM-5 molecular sieve provided in the present application, tetraethyl orthosilicate and trichloroacetic acid are utilized to perform dealumination and silicon-supplementation reaction on the ZSM-5 molecular sieve, which improves the silicon-aluminum ratio of the intermediate, thereby enhancing the stability of catalyst; and then melamine and metal salt are loaded on the intermediate in the form of a complex. Since the metal-melamine complex has a high stability and is not prone to loss, the metal loading rate is increased. After drying and roasting, the metal modified ZSM-5 molecular sieve catalyst prepared has good catalytic activity, light olefins selectivity and stability. Furthermore, the preparation process is simple and easy to operate; has mild conditions; and is friendly to environment, and beneficial to practical industrial production and application.

The metal modified ZSM-5 molecular sieve catalyst provided in the present application, which is prepared using the above-mentioned preparation method, has the advantages of good catalytic activity, light olefin selectivity and stability and so on, and can be applied to the production of light olefins, thereby enhancing the yield of light olefins.

### BRIEF DESCRIPTION OF DRAWINGS

FIG. 1 shows a nitrogen adsorption-desorption curve graph of a modified ZSM-5 molecular sieve catalyst in Embodiment 1.

### DESCRIPTION OF EMBODIMENTS

The specific embodiments listed below are only intended to describe the principles and characteristics of the present application, and the examples given are only used to explain the present application, not to limit the scope of the present application. Based on the embodiments of the present application, all other embodiments obtained by ordinary skilled persons in the field without creative labor fall within the protection scope of the present application.

The preparation method of the metal modified ZSM-5 molecular sieve catalyst provided in the present application includes the following steps: mixing a ZSM-5 molecular sieve, tetraethyl orthosilicate, trichloroacetic acid, and water for reaction, and filtering to obtain an intermediate; mixing the intermediate, melamine solution and metal salt solution; and drying, roasting, and obtaining the metal modified ZSM-5 molecular sieve catalyst.

After research and analysis, the applicant believes that in the preparation process of the metal modified ZSM-5 molecular sieve catalyst, utilizing tetraethyl orthosilicate and trichloroacetic acid to perform the dealumination and silicon-supplementation reaction on the ZSM-5 molecular sieve first, is beneficial to improving the silicon-aluminum ratio of the intermediate, further enhancing the thermal stability of intermediate while maintaining its higher crystallinity, and prolonging the service life of the catalyst. In addition, in the metal modified ZSM-5 molecular sieve catalyst, the performance of reaction and the selectivity of olefin products vary with different metal loading rate. The present application makes melamine and metal salt to load on the intermediate in the form of complex, the metal-melamine complex has high stability and is not prone to loss, thereby enhancing the metal loading ratio. Thus, the activity, stability and selectivity to light olefins of the catalyst are improved. The method of the present application also has the advantages of simple preparation process, easiness for operation, mild conditions and friendliness to the environment, and is beneficial to practical industrial production and application.

In the present application, the selected ZSM-5 molecular sieve includes, but is not limited to, a ZSM-5 molecular sieve with a silicon-aluminum ratio of 20-300. The selected ZSM-5 molecular sieve can be obtained by conventional methods, such as commercial purchase.

It should be noted that in the present application, a mass of ZSM-5 molecular sieve refers to a dry base mass of ZSM-5 molecular sieve.

In the present application, the ZSM-5 molecular sieve, tetraethyl orthosilicate, trichloroacetic acid and the water are mixed to obtain a mixed solution, and the mixed solution is subjected to the dealumination and silicon-enrichment reaction under certain conditions to obtain the intermediate, where the solid-liquid mass ratio of the mixed liquid is 1:(10-15).

Where, the dealumination and silicon-supplementation reaction refers to isomorphous replacement of aluminum atoms of the ZSM-5 molecular sieve skeleton with silicon atoms of tetraethyl orthosilicate to enhance the silica-aluminum ratio of the intermediate, thereby improving the thermal stability of intermediate.

The present application does not limit the process of mixing the ZSM-5 molecular sieve, tetraethyl orthosilicate, trichloroacetic acid and water for reaction. For example, trichloroacetic acid and water may be mixed first to form trichloroacetic acid solution; and then the ZSM-5 molecular sieve and the trichloroacetic acid solution may be mixed to undergo dealumination reaction, followed by adding tetraethyl orthosilicate and mixing to undergo silicon-supplementation reaction to obtain the intermediate.

The process of mixing trichloroacetic acid and water to form the trichloroacetic acid solution is carried out under stirring at normal temperature; in particular, it may be stirred at a temperature of 20° C.-30° C. for 10 min-30 min.

The ZSM-5 molecular sieve is first mixed and reacts with the trichloroacetic acid solution to remove part of aluminum from the ZSM-5 molecular sieve skeleton. At the same time of the dealumination, there are holes left at the location where the aluminum atoms originally exist. Such holes on the one hand increase the diameter of the pore structure of molecular sieve, and on the other hand may affect the stability of molecular sieve. At this case, tetraethyl orthosilicate is added to fill the holes with silicon atoms. The diameter of the pore structure of molecular sieve surface can be reduced because the bond length of Si—O is smaller than that of Al—O. Through such process of the dealumination and silicon-supplementation, the silicon-aluminum ratio of the product is improved while the retention rate of the crystallinity is ensured, thereby improving the thermal stability of the product.

Further, by controlling the conditions of the dealumination and silicon-supplementation reaction, the shrinkage degree of pore structure on the molecular sieve surface may be controlled, so that the ZSM-5 molecular sieve with different shrinkage degrees of surface pore size can be obtained. In the present application, by adjusting the conditions of the dealumination and silicon-supplementation reaction, the molecular sieve skeleton may generate mesopores via the dealumination and silicon-supplementation reaction, which is beneficial to the mass transfer of reaction materials and slows down the generation of coking and carbon deposition in the subsequent application of catalysis, thereby avoiding the reduction of catalyst activity and improving the stability of the catalyst.

In the above dealumination and silicon-supplementation reaction, a reaction temperature will affect the shrinkage degree of pore structure on the molecular sieve surface. This is because the reaction temperature will directly affect a reaction rate. If the temperature is too high, the rate reaction of dealumination and silicon-supplementation will be faster, making it difficult to control the pore structure of the molecular sieve, which is not conductive to keeping a retention degree of the skeleton structure of molecular sieve. If the temperature is too low, the reaction rate of dealumination and silicon-supplementation will be slow, which is not conductive to the subsequent reaction. In an implementation of the present application, the temperature of the dealumination and silicon-supplementation reaction is 80° C.

The aluminization and silicon-supplementation reaction can be carried out under stirring, where the time of the dealumination reaction is 10 min-30 min, and the time of the silicon-supplementation reaction is 20 min-60 min.

In the present application, the reaction conditions of dealumination and silicon-supplementation reaction are mild. In the process of dealumination and silicon-supplementation reaction, the aluminum atoms of the molecular sieve skeleton are selectively replaced with silicon atoms, not only maintaining the skeleton structure of ZSM-5 zeolite molecular sieve, but also obtaining the intermediate with mesopores, which improves the preparation efficiency and ensures the product quality. In the subsequent application in catalysis, it is beneficial to the mass transfer of reaction materials and slows down the generation of coking and carbon deposition.

After the ZSM-5 molecular sieve, tetraethyl orthosilicate, trichloroacetic acid and water are mixed for reaction, the intermediate is obtained by solid-liquid separation. The process of solid-liquid separation may be carried out by conventional separation methods in the art, such as suction filtration. In particular, a reaction product may be placed in a suction flask, and the pressure in the suction flask is reduced by using a pumping pump to achieve the purpose of solid-liquid separation. The solid phase obtained by the suction filtration is washed to obtain the intermediate.

The ratio of the ZSM-5 molecular sieve, tetraethyl orthosilicate and trichloroacetic acid may also affect the silicon-aluminum ratio of the resultant intermediate and the shrinkage degree of the pore structure of the molecular sieve surface. If the addition amount of trichloroacetic acid is too large, it will cause higher proportion of aluminum atoms to be removed from the ZSM-5 molecular sieve, and lead to too large diameter of the formed pore structure, which is not beneficial to the skeleton structure of molecular sieve. If the addition amount of trichloroacetic acid and tetraethyl orthosilicate is too small, it is not beneficial to the formation of mesoporous structure and the increase of the silicon-aluminum ratio. If too much tetraethyl orthosilicate is added, it will lead to waste of raw materials. In an implementation of the present application, the mass ratio of trichloroacetic acid to ZSM-5 molecular sieve is (0.8-3.5):1, such as 0.8:1, 0.9:1, 1:1, 1.5:1, 2:1, 2.5:1, 3:1, 3.5:1, or a range consisting of any two of them. The mass ratio of tetraethyl orthosilicate to ZSM-5 molecular sieve is (0.2-0.6):1, such as 0.2:1, 0.3:1, 0.5:1, 0.5:1, 0.6:1, or a range consisting of any two of them.

By mixing the intermediate, the melamine solution and the metal salt solution, the metal ions in the metal salt solution react with melamine in the melamine solution to form a metal-melamine complex, which is loaded on the intermediate. Due to the high stability of metal-melamine complex, it is not easy for the metal-melamine complex to lose in the subsequent solid-liquid separation process, thereby improving the metal loading ratio.

The present application does not limit the mixing mode of the intermediate, the melamine solution and the metal salt solution. In order to ensure that the metal-melamine complex can be uniformly loaded on the intermediate, the process of mixing the intermediate, the melamine solution and the metal salt solution includes: first mixing the intermediate and the melamine solution under stirring for 10 min-30 min so as to make them uniformly mixed, and obtaining a mixed solution; and mixing the mixed solution with the metal salt solution to allow the metal ions in the metal salt solution to be uniformly loaded on the intermediate in the form of metal-melamine complex under the action of the melamine solution.

In the present application, the melamine solution includes melamine and an organic solvent. The melamine is dissolved in the organic solvent, and then the intermediate and the melamine solution are mixed. By the introduction of the organic solvent, the dispersion of melamine and the intermediate may be improved, melamine may be uniformly dispersed on the surface of the intermediate under the effect of organic solvent, which also facilitates subsequent sufficient contact of melamine with the metal salt solution.

In the melamine solution, the mass ratio of the organic solvent to melamine is (0.4-1):1, such as 0.4:1, 0.5:1, 0.6:1, 0.7:1, 0.8:1, 0.9:1, 1:1, or a range consisting of any two of them.

The organic solvent is selected from at least one of acetic acid and ethylene glycol.

The melamine solution also contains water, which can further improve the dispersion of melamine.

The mass ratio of melamine to the ZSM-5 molecular sieve is (0.03-0.3):1, such as 0.03:1, 0.05:1, 0.08:1, 0.1:1, 0.12:1, 0.15:1, 0.2:1, 0.25:1, 0.3:1, or a range consisting of any two of them.

After the intermediate, the melamine solution and the metal salt solution are mixed for reaction, the reaction product is obtained. After solid-liquid separation, drying and roasting of the reaction product, the metal modified ZSM-5 molecular sieve catalyst is obtained.

The solid-liquid separation is intended to collect the solid phase in the reaction product. The solid-liquid separation process may be performed by using the conventional separation methods in the art, such as filtration or suction filtration.

The drying of the solid phase is intended to remove excess water from the surface of the solid phase, and the drying conditions include: the temperature is 100° C.-110° C., such as 100° C., 105° C., 110° C. or a range consisting of any two of them; the time is 8h-14h, such as 8h, 9h, 10h, 11h, 12h, 13h, 14h, or a range consisting of any two of them.

The roasting is intended to enable the metal-melamine complex loaded on the surface of intermediate to form a metal oxide, the roasting conditions include: the temperature is 480° C.-580° C., such as 480° C., 500° C., 550° C., 580° C. or a range consisting of any two of them; the time is 1h-4h, such as 1h, 2h, 3h, 4h, or a range consisting of any two of them.

In the present application, the metal salt solution includes the metal salt and water, where a metal element in the metal salt is selected from at least one of Fe, Cu, Ti, Zr, Mn and Zn. Based on a total mass of the metal modified ZSM-5 molecular sieve catalyst as 100%, the mass ratio of the metal element is 0.02-10%, where the mass of the metal element is calculated based on a corresponding metal oxide. The mass ratio of the ZSM-5 molecular sieve is 92-99.98%.

The present application also includes a post-treatment of the roasting product, the post-treatment includes subjecting the roasting product to compression molding at 20 MPa and screening to obtain 20-40 mesh particles, i.e. the metal modified ZSM-5 molecular sieve catalyst.

The present application also provides a metal modified ZSM-5 molecular sieve catalyst, which is prepared by the above-mentioned preparation method. It has the advantages of good catalytic activity, light olefins selectivity and stability, and can be applied to the production of light olefins, thereby enhancing the yield of light olefins.

The application also provides a preparation method of light olefins, including: allowing alkane material to react under the catalytic action of the above-mentioned metal modified ZSM-5 molecular sieve catalyst to produce light olefins. Where, the alkane material includes C6-C10 alkane.

The preparation process of the above-mentioned light olefins may be carried out in a fixed bed, for example in a continuous flow fixed bed reactor. Specifically, the catalyst may be filled in a reactor, the reactor is purged with an inert gas, and then the alkane material flows through the catalyst for reaction. The reaction conditions are as follows: the temperature is 630° C., the weight hourly space velocity of feed is 6h⁻¹, and the alkane material may enter into the reactor by means of pumping.

The present application is further explained by specific embodiments and comparative embodiments below. Unless otherwise specified, the reagents, materials and instruments used below are conventional reagents, conventional materials and conventional instruments respectively, which may be obtained by commercial purchase; and the reagents and materials involved may also be synthesized by conventional synthesis methods.

In the following embodiments and comparative embodiments, the raw materials used include: an industrial ZSM-5 molecular sieve (loss on ignition 8.9 wt %), sourced from Lanzhou Petrochemical Catalyst Plant;
melamine, acetic acid, ethylene glycol, trichloroacetic acid, tetraethyl orthosilicate, ferric chloride hexahydrate, copper chloride dihydrate, titanium sulfate, zirconium chloride, manganese chloride tetrahydrate, and zinc chloride, where all of above materials are analytical pure, and purchased from Sinopharm Chemical Reagent Co., Ltd.

The following embodiments and comparative embodiments are evaluated using the following methods.

The pore structure of catalyst is determined by the low temperature nitrogen adsorption-desorption method; and the content of metal on the catalyst is determined by the fluorescence method. (Analytical methods refer to “Analytical Methods for Petrochemical Industry (RIPP Experimental Methods)”, edited by Yang Cuiding et al., Science Press, 1990).

The evaluation of catalyst reaction performance is performed in a continuous flow fixed bed reactor, where, the loading amount of catalyst in the reactor is 5 g, and the reactor is filled with quartz cotton at both ends; before the reaction, the reactor is purged with N₂ for 30 min, with a carrier gas being N₂; the reaction material is n-hexane, which is pumped into the reactor by a micropump; the reaction temperature is 630° C., the weight hourly space velocity of feed is 6h⁻¹, and the reaction products are analyzed online by SP-3420 gas chromatography; and the catalyst service life data is based on the time from the beginning of feed to the conversion rate falling to 90% as a reference.

Where, taking n-hexane as the raw material, the conversion rate of n-hexane and the selectivity of product distribution are calculated respectively according to the following formula:

$$conversion rate = \frac{{w}_{in} - {w}_{out}}{{w}_{in}} \times 100 \%$$

and

$$Selectivity = \frac{{w}_{{C}_{x} {H}_{y} out}}{{Σ}_{{C}_{x} {H}_{y} out}} \times 100 \% ,$$

where wᵢₙ is a mass fraction of n-hexane in the reaction raw material, wₒᵤₜ is a mass fraction of n-hexane in the reaction product, and w_{CxHyout} is a mass fraction of a certain substance in the product. Modified metal retention rate=the amount of metal loaded by catalyst/the total amount of metal added.

The evaluation results are shown in Table 1.

### Embodiment 1

(1) 1.8 g melamine, 0.72 g ethylene glycol and 40 g deionized water were mixed under stirring at 25° C. for 10 min to be uniform, obtaining a melamine solution (slurry a);
(2) 20 g ZSM-5 molecular sieve with a silicon-aluminum ratio of 35 (dry mass), 16 g trichloroacetic acid and 200 g deionized water were mixed, and stirred at 80° C. for 20 min, then 4 g tetraethyl orthosilicate was added and stirred for 40 min, and subjected to suction filtering and washing until neutral to obtain an intermediate (sample b);
(3) The sample b was added to the slurry a for mixing, followed by stirring for 20 min to obtain a slurry c;
(4) 2.89 g ferric chloride hexahydrate was weighed and added to the slurry c and stirred for 30 min to obtain a slurry d; the slurry d was filtered to obtain a solid phase; and then the solid phase was dried at 100° C. for 12h and roasted at 550° C. for 4h, obtaining a metal Fe modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C1 for evaluating the reaction performance of the catalyst.

### Embodiment 2

(1) 0.6 g melamine, 0.24 g acetic acid and 40 g deionized water were mixed under stirring at 25° C. for 10 min to be uniform, obtaining a melamine solution (slurry a);
(2) 20 g ZSM-5 molecular sieve with a silicon-aluminum ratio of 20 (dry mass), 43 g trichloroacetic acid and 250 g deionized water were mixed, and stirred at 80° C. for 20 min, then 8 g tetraethyl orthosilicate was added and stirred for 40 min, and subjected to suction filtering and washing until neutral to obtain an intermediate (sample b);
(3) The sample b was added to the slurry a for mixing, followed by stirring for 20 min to obtain a slurry c;
(4) 0.02 g ferric chloride hexahydrate was weighed and added to the slurry c and stirred for 30 min to obtain a slurry d; the slurry d was filtered to obtain a solid phase, and then the solid phase was dried at 100° C. for 12h and roasted at 550° C. for 4h, obtaining a metal Fe modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C2 for evaluating the reaction performance of the catalyst.

### Embodiment 3

(1) 3 g melamine, 1.8 g acetic acid and 40 g deionized water were mixed under stirring at 25° C. for 10 min to be uniform, obtaining a melamine solution (slurry a).
(2) 20 g ZSM-5 molecular sieve with a silicon-aluminum ratio of 100 (dry mass), 30 g trichloroacetic acid and 200 g deionized water were mixed, and stirred at 80° C. for 20 min, then 6 g tetraethyl orthosilicate was added and stirred for 40 min, and subjected to suction filtering and washing until neutral to obtain an intermediate (sample b);
(3) The sample b was added to the slurry a for mixing, and stirring for 20 min to obtain a slurry c;
(4) 2.11 g copper chloride dihydrate was weighed and added to the slurry c and stirred for 30 min to obtain a slurry d; the slurry d was filtered to obtain a solid phase, and then the solid phase was dried at 100° C. for 12h and roasted at 550° C. for 4h, obtaining a metal Cu modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C3 for evaluating the reaction performance of the catalyst.

### Embodiment 4

(1) 4.8 g melamine, 3.4 g ethylene glycol and 40 g deionized water were mixed under stirring at 25° C. for 10 min to be uniform, obtaining a melamine solution (slurry a);
(2) 20 g ZSM-5 molecular sieve with a silicon-aluminum ratio of 200 (dry mass), 36 g trichloroacetic acid and 300 g deionized water were mixed, and stirred at 80° C. for 20 min, then 8 g tetraethyl orthosilicate was added and stirred for 40 min, and subjected to suction filtering and washing until neutral to obtain an intermediate (sample b);
(3) The sample b was added to the slurry a for mixing, followed by stirring for 20 min to obtain a slurry c;
(4) 3.38 g copper chloride dihydrate was weighed and added to the slurry c and stirred for 30 min to obtain a slurry d; the slurry d was filtered to obtain a solid phase, and then the solid phase was dried at 100° C. for 12h and roasted at 550° C. for 4h, obtaining a metal Cu modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C4 for evaluating the reaction performance of the catalyst.

### Embodiment 5

(1) 0.6 g melamine, 0.5 g ethylene glycol and 40 g deionized water were mixed under stirring at 25° C. for 10 min to be uniform, obtaining a melamine solution (slurry a);
(2) 20 g ZSM-5 molecular sieve with a silicon-aluminum ratio of 250 (dry mass), 40 g trichloroacetic acid and 200 g deionized water were mixed, and stirred at 80° C. for 20 min; and then 10 g tetraethyl orthosilicate was added and stirred for 40 min and subjected to suction filtering and washing until neutral to obtain an intermediate (sample b);
(3) The sample b was added to the slurry a for mixing, followed by stirring for 20 min to obtain a slurry c;
(4) 1.00 g titanium sulfate was weighed and added to the slurry c and stirred for 30 min to obtain a slurry d; the slurry d was filtered to obtain a solid phase; and then the solid phase was dried at 100° C. for 12h and roasted at 550° C. for 4h, obtaining a metal Ti modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C5 for evaluating the reaction performance of the catalyst.

### Embodiment 6

(1) 2.4 g melamine, 2.4 g acetic acid and 40 g deionized water were mixed under stirring at 25° C. for 10 min to be uniform, obtaining a melamine solution (slurry a);
(2) 20 g ZSM-5 molecular sieve with a silicon-aluminum ratio of 300 (dry mass), 42 g trichloroacetic acid and 200 g deionized water were mixed, and stirred at 80° C. for 20 min, then 12 g tetraethyl orthosilicate was added and stirred for 40 min, and subjected to suction filtering and washing until neutral to obtain an intermediate (sample b);
(3) The sample b was added to the slurry a for mixing, followed by stirring for 20 min to obtain a slurry c;
(4) 4.00 g titanium sulfate was weighed and added to the slurry c and stirred for 30 min to obtain a slurry d; the slurry d was filtered to obtain a solid phase, and then the solid phase was dried at 100° C. for 12h and roasted at 550° C. for 4h, obtaining a metal Ti modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C6 for evaluating the reaction performance of the catalyst.

### Embodiment 7

(1) 0.3 g melamine, 0.24 g acetic acid and 40 g deionized water were mixed under stirring at 25° C. for 10 min to be uniform, obtaining a melamine solution (slurry a);
(2) 20 g ZSM-5 molecular sieve with a silicon-aluminum ratio of 35 (dry mass) and 70 g trichloroacetic acid were added to 200 g deionized water, and stirred at 80° C. for 20 min, then 8 g tetraethyl orthosilicate was added and stirred for 40 min and subjected to suction filtering and washing until neutral to obtain an intermediate (sample b);
(3) The sample b was added to the slurry a for mixing, followed by stirring for 20 min to obtain a slurry c;
(4) 0.26 g zirconium chloride was weighed and added to the slurry c and stirred for 30 min to obtain a slurry d; the slurry d was filtered to obtain a solid phase; and then the solid phase was dried at 100° C. for 12h and roasted at 550° C. for 4h, obtaining a metal Zr modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C7 for evaluating the reaction performance of the catalyst.

### Embodiment 8

(1) 1.2 g melamine, 0.96 g ethylene glycol and 40 g deionized water were mixed under stirring at 25° C. for 10 min to be uniform, obtaining a melamine solution (slurry a);
(2) 20 g ZSM-5 molecular sieve with a silica-aluminum ratio of 100 (dry mass) and 60 g trichloroacetic acid were added to 200 g deionized water, and stirred at 80° C. for 20 min; then 6 g tetraethyl orthosilicate was added and stirred for 40 min and subjected to suction filtering and washing until neutral to obtain an intermediate (sample b);
(3) The sample b was added to the slurry a for mixing, followed by stirring for 20 min to obtain a slurry c;
(4) 1.02 g zirconium chloride was weighed and added to the slurry c and stirred for 30 min to obtain a slurry d; the slurry d was filtered to obtain a solid phase; and then the solid phase was dried at 100° C. for 12h and roasted at 550° C. for 4h, obtaining a metal Zr modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C8 for evaluating the reaction performance of the catalyst.

### Embodiment 9

(1) 4.2 g melamine, 3.78 g ethylene glycol and 40 g deionized water were mixed under stirring at 25° C. for 10 min to be uniform, obtaining a melamine solution (slurry a);
(2) 20 g ZSM-5 molecular sieve with a silicon-aluminum ratio of 300 (dry mass) and 50 g trichloroacetic acid were added to 200 g deionized water and stirred at 80° C. for 20 min; and then 10 g tetraethyl orthosilicate was added and stirred for 40 min and subjected to suction filtering and washing until neutral to obtain an intermediate (sample b);
(3) The sample b was added to the slurry a for mixing, followed by stirring for 20 min to obtain a slurry c;
(4) 5.04 g manganese chloride tetrahydrate was weighed and added to the slurry c and stirred for 30 min to obtain a slurry d; the slurry d was filtered to obtain a solid phase; and then the solid phase was dried at 100° C. for 12h and roasted at 550° C. for 4h, obtaining a metal Mn modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C9 for evaluating the reaction performance of the catalyst.

### Embodiment 10

(1) 6 g melamine, 6 g ethylene glycol and 40 g deionized water were mixed under stirring at 25° C. for 10 min to be uniform, obtaining a melamine solution (slurry a);
(2) 20 g ZSM-5 molecular sieve with a silicon-aluminum ratio of 35 (dry mass) and 56 g trichloroacetic acid were added to 200 g deionized water and stirred at 80° C. for 20 min; and then 12 g tetraethyl orthosilicate was added and stirred for 40 min and subjected to suction filtering and washing until neutral to obtain an intermediate (sample b);
(3) The sample b was added to the slurry a for mixing, followed by stirring for 20 min to obtain a slurry c;
(4) 4.18 g zinc chloride was weighed and added to the slurry c and stirred for 30 min to obtain a slurry d; the slurry d was filtered to obtain a solid phase; and then the solid phase was dried at 100° C. for 12h and roasted at 550° C. for 4h, obtaining a metal Zn modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C10 for evaluating the reaction performance of the catalyst.

### Embodiment 11

(1) 1.8 g melamine, 0.72 g ethylene glycol and 40 g deionized water were mixed under stirring at 25° C. for 10 min to be uniform, obtaining a melamine solution (slurry a);
(2) 20 g ZSM-5 molecular sieve with a silica-aluminum ratio of 35 (dry mass), 100 g trichloroacetic acid and 100 g deionized water were mixed, and stirred at 80° C. for 20 min; and then 20 g tetraethyl orthosilicate was added and stirred for 40 min and subjected to suction filtering and washing until neutral to obtain an intermediate (sample b);
(3) The sample b was added to the slurry a for mixing, followed by stirring for 20 min to obtain a slurry c;
(4) 2.89 g ferric chloride hexahydrate was weighed and added to the slurry c and stirred for 30 min to obtain a slurry d; the slurry d was filtered to obtain a solid phase; and then the solid phase was dried at 100° C. for 12h and roasted at 550° C. for 4h, obtaining a metal Fe modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C11 for evaluating the reaction performance of the catalyst.

### Embodiment 12

(1) 1.8 g melamine, 0.72 g ethylene glycol and 40 g deionized water were mixed under stirring at 25° C. for 10 min to be uniform, obtaining a melamine solution (slurry a);
(2) 20 g ZSM-5 molecular sieve with a silicon-aluminum ratio of 35 (dry mass), 16 g trichloroacetic acid and 200 g deionized water were mixed, and stirred at 80° C. for 20 min; and then 4 g tetraethyl orthosilicate was added and stirred for 40 min, and subjected to suction filtering and washing until neutral to obtain an intermediate (sample b);
(3) The sample b was added to the slurry a for mixing, followed by stirring for 20 min to obtain a slurry c;
(4) 2.89 g ferric chloride hexahydrate was weighed and added to the slurry c and stirred for 30 min to obtain a slurry d; the slurry d was filtered to obtain a solid phase; and then the solid phase was dried at 100° C. for 12h and roasted at 550° C. for 4h, obtaining a metal Fe modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst C12 for evaluating the reaction performance of the catalyst.

### Comparative Embodiment 1

(1) 20 g ZSM-5 molecular sieve with a silicon-aluminum ratio of 300 (dry mass) and 40 g deionized water were mixed under stirring at 25° C. for 10 min to be uniform, obtaining a melamine solution (slurry a);
(2) 2.89 g ferric chloride hexahydrate was added to the slurry a, and stirred for 30 min to obtain a slurry b; the slurry b was filtered to obtain a solid phase, and then the solid phase was dried at 100° C. for 12h and roasted at 550° C. for 4 h, obtaining a metal Fe modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst D1 for evaluating the reaction performance of the catalyst.

### Comparative Embodiment 2

20 g ZSM-5 molecular sieve with a silicon-aluminum ratio of 10 (dry mass), 1.02 g titanium sulfate and 12 g deionized water were mixed at 25° C. to be uniform, followed by metal Ti impregnation modification, and drying at 100° C. for 12h and roasted at 550° C. for 4h, thereby obtaining a metal Ti modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst D2 for evaluating the reaction performance of the catalyst.

### Comparative Embodiment 3

10 g ZSM-5 molecular sieve with a silicon-aluminum ratio of 30 (dry mass) and 0.01 g ZrO₂ particles were mixed fully, and 20 g deionized water was slowly added and then stirred thoroughly; the solid-liquid mixture was transferred to a high-temperature reactor and subjected to a reaction at 240° C. for 6h, where the reaction pressure was a normal pressure; and the reactor was taken out, cooled to room temperature in air, and opened and dried at 140° C. for 10h, obtaining a metal Zr modified ZSM-5 molecular sieve catalyst powder prepared by a hydrothermal method;
the catalyst powder was subjected to compression molding at 20 MPa and screened to obtain 20-40 mesh particles, thereby obtaining the metal modified ZSM-5 molecular sieve catalyst D3 for evaluating the reaction performance of the catalyst.

### Comparative Embodiment 4

6 g melamine, 6 g ethylene glycol and 40 g deionized water were mixed under stirring at 25° C. for 10 min to be uniform, obtaining a melamine solution (slurry a); then 20 g ZSM-5 molecular sieve with a silicon-aluminum ratio of 35 (dry mass) was added to the slurry a for mixing, followed by stirring for 20 min to obtain a slurry b; then 2.89 g ferric chloride hexahydrate was weighed and added to the slurry b and stirred for 30 min to obtain a slurry c; finally, the slurry c was filtered to obtain a solid phase, and then the solid phase was dried at 100° C. for 12h and roasted at 550° C. for 4h, obtaining a metal Zn modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst D4 for evaluating the reaction performance of the catalyst.

### Comparative Embodiment 5

20 g ZSM-5 molecular sieve with a silicon-aluminum ratio of 35 (dry mass) and 56 g trichloroacetic acid were added to 200 g deionized water and stirred at 80° C. for 20 min, and then 12 g tetraethyl orthosilicate was added and stirred for 40 min, and subjected to suction filtering and washing until neutral to obtain an intermediate (sample a); then 2.89 g ferric chloride hexahydrate, 12 g deionized water and the sample a were mixed to be uniform and subjected to metal Fe impregnation modification, and finally dried at 100° C. for 12h and roasted at 550° C. for 4h, obtaining the metal Fe modified ZSM-5 molecular sieve catalyst powder;
the catalyst powder was subjected to compression molding at 20 MPa and screened to obtain 20-40 mesh particles, thereby obtaining the metal modified ZSM-5 molecular sieve catalyst D5 for evaluating the reaction performance of the catalyst.

TABLE 1

| | Modified | | | | | | |
|---|---|---|---|---|---|---|---|
| | Metal | | | Diene | | | Service |
| | Retention | | | (Ethylene + | | Carbon | Life of |
| | Rate | Ethylene | Propylene | Propylene) | Conversion | Deposition | Catalyst |
| Catalyst | % | Selectivity % | Selectivity % | Selectivity % | Rate % | Rate (wt %/h) | h |
| C1 | 99.9 | 23.2 | 26.7 | 49.9 | 99.7 | 0.010 | 85.3 |
| C2 | 99.8 | 22.0 | 25.1 | 47.1 | 99.2 | 0.011 | 80.5 |
| C3 | 99.8 | 22.7 | 25.4 | 48.1 | 99.5 | 0.010 | 80.2 |
| C4 | 99.9 | 22.6 | 25.3 | 47.9 | 99.4 | 0.013 | 80.1 |
| C5 | 100 | 22.6 | 25.2 | 47.8 | 99.5 | 0.011 | 80.6 |
| C6 | 99.7 | 22.8 | 25.4 | 48.2 | 99.6 | 0.013 | 80.1 |
| C7 | 99.9 | 22.7 | 25.5 | 48.2 | 99.4 | 0.011 | 80.3 |
| C8 | 99.9 | 22.9 | 26.3 | 49.2 | 99.8 | 0.012 | 82.3 |
| C9 | 99.8 | 22.8 | 25.9 | 48.7 | 99.4 | 0.011 | 82.5 |
| C10 | 99.7 | 23.1 | 26.6 | 49.7 | 99.6 | 0.013 | 80.1 |
| C11 | 99.8 | 22.5 | 25.4 | 47.9 | 99.4 | 0.017 | 75.2 |
| C12 | 99.9 | 22.6 | 25.5 | 48.1 | 99.5 | 0.015 | 78.5 |
| D1 | 32.3 | 21.4 | 21.1 | 42.5 | 99.2 | 0.021 | 52.1 |
| D2 | 99.4 | 21.6 | 25.2 | 46.8 | 99.4 | 0.024 | 55.2 |
| D3 | 98.5 | 21.5 | 25.1 | 46.6 | 99.3 | 0.022 | 59.5 |
| D4 | 99.9 | 21.7 | 22.5 | 44.2 | 99.5 | 0.028 | 48.9 |
| D5 | 99.4 | 21.1 | 22.7 | 43.8 | 99.4 | 0.025 | 51.8 |
| Blank | / | 17.8 | 19.1 | 36.9 | 92.5 | 0.020 | 23.2 |
| Agent* | | | | | | | |

In the table: Blank agent* is unmodified ZSM-5 molecular sieve sample; “/” represents no metal loading.

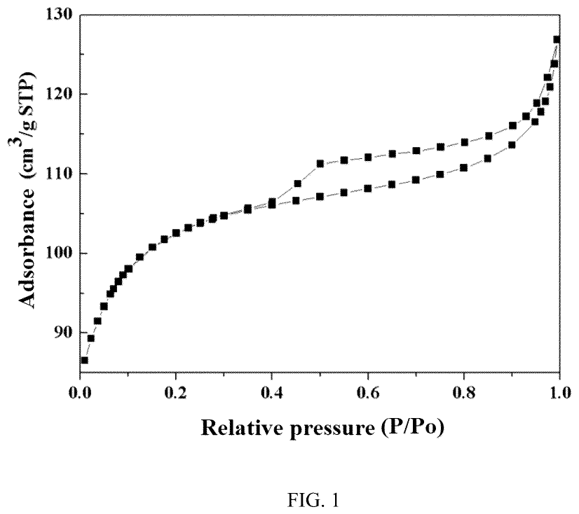

FIG. 1 shows the nitrogen adsorption-desorption curve graph of the modified ZSM-5 molecular sieve catalyst in Embodiment 1. According to FIG. 1, it can be seen that the modified ZSM-5 molecular sieve catalyst in Embodiment 1 contains a mesoporous structure.

According to Table 1, it can be seen that when the metal modified ZSM-5 molecular sieve catalyst is prepared by the method provided in the present application, there is hardly the loss of metal and the metal retention rate is high, thereby improving the metal loading rate in the catalyst. Moreover, each carbon deposition rate is below 0.02 wt %/h and the service life of catalyst is above 80h, thereby having a lower carbon deposition rate and longer catalyst life. When the catalyst is applied to the preparation of light olefins, the selectivity of light olefins can be significantly improved, thereby increasing the yield of light olefins.

The above describes in detail the preferred specific embodiments and the test validation of the present application. It should be understood that the skilled in the art can make various modifications and changes in accordance with the conception of the present application without creative effort. Therefore, the technical solutions, which can be obtained by persons of ordinary skill in the art on the basis of the existing technology through logical analysis, reasoning or limited experiments according to the conception of the present application, shall fall within the scope of protection determined by the claims.

What is claimed is:

1. A preparation method of metal modified ZSM-5 molecular sieve catalyst, comprising the following steps:
mixing a ZSM-5 molecular sieve, tetraethyl orthosilicate, trichloroacetic acid, and water for reaction, and filtering to obtain an intermediate;
mixing the intermediate, a melamine solution and a metal salt solution, and drying and roasting to obtain the metal modified ZSM-5 molecular sieve catalyst.

2. The preparation method according to claim 1, wherein a process of mixing the intermediate, a melamine solution and a metal salt solution comprises: first mixing the intermediate and the melamine solution, and stirring for 10 min-30 min to obtain a mixed solution, and then mixing the mixed solution and the metal salt solution.

3. The preparation method according to claim 1, wherein the melamine solution comprises melamine and an organic solvent, wherein a mass ratio of the organic solvent to the melamine is (0.4-1):1.

4. The preparation method according to claim 2, wherein the melamine solution comprises melamine and an organic solvent, wherein a mass ratio of the organic solvent to the melamine is (0.4-1):1.

5. The preparation method according to claim 3, wherein a mass ratio of the melamine to the ZSM-5 molecular sieve is (0.03-0.3):1.

6. The preparation method according to claim 4, wherein a mass ratio of the melamine to the ZSM-5 molecular sieve is (0.03-0.3):1.

7. The preparation method according to claim 3, wherein the organic solvent is selected from at least one of acetic acid and ethylene glycol.

8. The preparation method according to claim 4, wherein the organic solvent is selected from at least one of acetic acid and ethylene glycol.

9. The preparation method according to claim 5, wherein the organic solvent is selected from at least one of acetic acid and ethylene glycol.

10. The preparation method according to claim 6, wherein the organic solvent is selected from at least one of acetic acid and ethylene glycol.

11. The preparation method according to claim 1, wherein a mass ratio of the trichloroacetic acid to the ZSM-5 molecular sieve is (0.8-3.5):1; and/or
a mass ratio of the tetraethyl orthosilicate to the ZSM-5 molecular sieve is (0.2-0.6):1.

12. The preparation method according to claim 1, wherein the reaction is performed at a temperature of 80° C. for 10 min-60 min.

13. The preparation method according to claim 1, wherein the drying is performed at a temperature of 100° C.-110° C. for 8h-14h; and/or
the roasting is performed at a temperature of 480° C.-580° C. for 1h-4h.

14. A metal modified ZSM-5 molecular sieve catalyst prepared by the preparation method according to claim 1.

15. The metal modified ZSM-5 molecular sieve catalyst according to claim 14, wherein a process of mixing the intermediate, a melamine solution and a metal salt solution comprises: first mixing the intermediate and the melamine solution, and stirring for 10 min-30 min to obtain a mixed solution, and then mixing the mixed solution and the metal salt solution.

16. A preparation method of light olefins, comprising: allowing alkane material to react under a catalytic action of the metal modified ZSM-5 molecular sieve catalyst according to claim 14 to obtain light olefins.
